(19) United States
(12) Patent Application Publication
(10) Pub. No.: US 2016/0169860 A1
(43) Pub. Date: Jun. 16, 2016

(54) METHOD FOR CALCULATING SWELLING PHENOMENON EVALUATION INDEX OF POLYMER AND SYSTEM USING SAME

(71) Applicant: LG CHEM, LTD., Seoul (KR)

(72) Inventors: Seungyup LEE, Daejeon (KR); Yonggoo SON, Daejeon (KR); Kyounghoon KIM, Daejeon (KR)

(21) Appl. No.: 14/906,168

(22) PCT Filed: Aug. 14, 2014
(86) PCT No.: PCT/KR2014/007566

§ 371 Date: Jan. 19, 2016

(30) Foreign Application Priority Data
Sep. 2, 2013 (KR) 10-2013-0104821

(51) Int. Cl. G01N 33/44; G01N 33/487

(52) U.S. Cl. CPC G01N 33/44; G01N 33/487

(57) ABSTRACT

The present invention relates to a method for calculating the swelling phenomenon evaluation index of a polymer and a system using the same and, more specifically, to a method for calculating the swelling phenomenon evaluation index of a polymer, wherein the method employs a solvent-polymer swelling parameter (hereinafter, S-PSP), which is a new method developed to quantitatively evaluate the swelling phenomenon of the polymer with respect to different solvents, and to a system using the same.

### TECHNICAL FIELD

The present invention relates to a method of calculating an evaluation index of polymer swelling, and a system using the same. More particularly, the present invention relates to a method of calculating an evaluation index of polymer swelling, based on Solvent-Polymer Swelling Parameter (hereinafter referred to as S-PSP), which is a novel concept of quantitatively accounting for polymer swelling in different solvents, and a system for calculating an evaluation index of polymer swelling, using the same.

### BACKGROUND ART

When exposed to a solvent, polymers undergo swelling due to the penetration of solvent molecules into inter-polymer chain spaces. Because polymer swelling greatly varies depending on various factors including polymer structures (crystal or non-crystal structures), molecular weights, molecular weight distribution, solvent properties, etc., there have been no methods of definitely evaluating polymer swelling.

To assess solubility or miscibility among different materials, intrinsic properties of such materials should be analyzed for similarity. There are various intrinsic properties that have effects on solubility or miscibility. Inter alia, solubility parameters, which express interaction between materials as quantitative values, are most common. That is, materials have respective intrinsic solubility parameters, and are well dissolved or miscible together if their solubility parameter values are similar.

Solubility parameters have been proposed and used on the basis of various theories and concepts. Among them, the Hansen Solubility Parameter (hereinafter referred to as “HSP”), developed by Dr. C. Hansen in 1967, is known to most accurately represent solubility properties. In the HSP, interaction between materials is considered in terms of the following three solubility parameters:

(1) solubility parameter generated by non-polar dispersion energy (ED)

(2) solubility parameter generated by polar energy due to a permanent dipole moment (δP)

(3) solubility parameter generated by energy within hydrogen bonds (δH)

As such, the HSP is widely used because it can provide information on intermolecular interaction in greater detail and thus can evaluate solubility or miscibility between materials more accurately and systemically than other solubility parameters.

HSP=(δD,δP,δH),(J/cm³)^{1/2} (1)

δTot=(δD²+δP²+δH²)^{1/2},(J/cm³)^{1/2} (2)

The HSP represents vector properties with magnitude and direction in the Hansen space defined by the three parameters as coordinates while δTot represents the magnitude of the HSP vector. HSP is measured in (J/cm³)^{1/2}. These HSP values can be calculated using the program HSPiP (Hansen Solubility Parameters in Practice) developed by the Dr. Hansen Group.

As mentioned above, two different materials are soluble with respect to each other when they are similar in HSP. Since HSP is a vector, the necessary condition for determining the similarity of HSP between two materials is that all the three HSP elements must be similar in magnitude and direction therebetween. Every material has its intrinsic HSP, and two different materials are miscible when they are similar in HSP. Like other solubility parameters, HSP was proposed on the concept that ‘a like likes a like.’

However, HSP alone has difficulty in accounting for the quantitative evaluation of polymer swelling. The necessity that arises from the limitation of HSP in precisely evaluating polymer swelling has inspired the present inventor to conceive a solvent-polymer swelling parameter (S-PSP), a novel concept of quantitatively accounting for the solvent penetration-caused polymer swelling, on the basis of the adjustment of the HSP of solvents, and to develop a novel method of calculating a swelling index of polymer swelling, using the Solvent-Polymer Swelling Parameter (S-PSP), whereby polymer swelling can definitely evaluated.

### DISCLOSURE

### Technical Problem

Accordingly, the present invention has been made keeping in mind the above problems occurring in the prior art, and an object of the present invention is to provide a novel method of calculating an evaluation index of polymer swelling using Solvent-Polymer Swelling Parameter (S-PSP), which is a new concept for quantitatively evaluating polymer swelling.

### Technical Solution

In accordance with an aspect thereof, the present invention provides a method of calculating an evaluation index of polymer swelling, the method comprising:

a) performing a swelling experiment with N different solvents to assess a degree of swelling of a polymer to be dissolved;

b) calculating a Solvent-Polymer Swelling Parameter (S-PSP), based on Hansen Solubility Parameters (HSPs) adjusted for the N different solvents employed in the swelling experiment of step a); and

c) calculating a Solvent-Polymer Swelling Parameter Distance (S-Distance) with regard to the N different solvents that are given the calculated S-PSP of step b) to identify polymer swelling.

In accordance with another aspect thereof, the present invention provides a system for calculating an evaluation index of polymer swelling, comprising:

an evaluation module for receiving data obtained by performing a swelling experiment with N different solvents to assess a degree of swelling of a polymer to be dissolved;

a data input module for receiving data obtained by calculating a Solvent-Polymer Swelling Parameter (S-PSP), based on Hansen Solubility Parameters (HSPs) adjusted for the N different solvents employed in the swelling experiment of the evaluation module; and

an identification module for receiving data obtained by calculating Solvent-Polymer Swelling Parameter Distance (S-Distance) with regard to the N different solvents that are given the calculated S-PSP of the data input module to identify polymer swelling.

### Advantageous Effects

Characterized by use of Solvent-Polymer Swelling Parameter (S-PSP), which is a property evaluation parameter allowing for the quantitative evaluation of polymer swelling in different solvents, the method of calculating an evaluation index of polymer swelling in accordance with the present invention can be effectively used for predicting the increment of volume or weight of polymer on swelling. Compared to conventional methods, the method of the present invention can accurately evaluate polymer swelling, which has great influence on polymer performance and properties. Thus, the present invention finds advantageous applications in developing polymeric materials and enhancing polymer-based process performance, and is expected to be effective for the systematic use and evaluation of polymeric materials.

### DESCRIPTION OF DRAWINGS

FIG. 1 is a graph illustrating correlation between the Solvent-Polymer Swelling Parameter (S-PSP) and the relative swelling amount of polymer in accordance with an embodiment of the present invention.

### BEST MODE

Below, a detailed description will be given of the present invention.

The present invention addresses a method of calculating an evaluation index of polymer swelling, comprising:

a) performing a swelling experiment with N different solvents to assess a degree of swelling of a polymer to be dissolved;

b) calculating a Solvent-Polymer Swelling Parameter (S-PSP), based on Hansen Solubility Parameters (HSPs) adjusted for the N different solvents employed in the swelling experiment of step a); and

c) calculating a Solvent-Polymer Swelling Parameter Distance (S-Distance) with regard to the N different solvents that are given the calculated S-PSP of step b) to identify swelling.

The assessing of a degree of swelling of a polymer of step a) may comprise measuring an increment of volume or weight of the polymer that swells due to the penetration of the solvent to the polymer.

In greater detail, the degree of swelling of a polymer may be measured as a relative amount of polymer swelling. The relative amount of polymer swelling may be a weight increment of the polymer on swelling in each of the N different solvents, divided by the highest increment thereamong.

The number N of solvents is not specifically limited so long as it is a natural number larger than zero. In a preferable embodiment, the number N is an integer of 3 to 20.

In the present invention, the calculating of a Solvent-Polymer Swelling Parameter (S-PSP) of step b) may be performed using the following Equations 1 to 3:

S-PSP{Ai}=(a0x(ADJ_D{Ai})^{b0}+a1x(ADJ_P{Ai})^{b1}+a2x(ADJ_H{Ai})^{b2})^{c} Equation 1

ADJ_D{Ai}=F(D{Ai}),ADJ_P{Ai}=F(P{Ai}),ADJ_H{Ai}=F(H{Ai}) Equation 2

F(X)=d0×exp(X/d1) or F(X)=d2×log₁₀((X+1)/d3) Equation 3

In Equation 1, Ai represents an i^{th} solvent of the N different solvents used in the swelling experiment; a0, a1, and a2 are each real numbers; b0, b1, and b2 are each real numbers; and c is a real number. In Equation 2, ADJ_D{Ai}, ADJ_P{Ai}, and ADJ_H{Ai} each represent adjusted Hansen Solubility Parameters wherein D{Ai}, P{Ai}, and H{Ai} are respectively solubility parameters generated by non-polar dispersion, by polar energy due to a permanent dipole moment, and by energy within hydrogen bonds, respectively, for a certain solvent Ai. In Equation 3, F(X) is a function for adjusting an HSP for solvent Ai, and d0, d1, d2, and d3 are each real numbers. In the equations, preferably, b0 is a real number of 0 to 3.5, b1 is a real number of 0 to 5.0, and b2 is a real number of 0 to 4.0.

In order to evaluate polymer swelling, the Solvent-Polymer Swelling Parameter was developed on the basis of the adjustment of Hansen Solubility Parameter for solvents.

In step c), the calculating of Solvent-Polymer Swelling Parameter Distance (S-Distance) to identify polymer swelling may comprise:

i) calculating a Solvent-Polymer Swelling Distance (S-Distance) according to the following Equation 4; and

ii) when the S-Distance calculated in step i) is larger than a cut-off value (a real number larger than zero), stopping the calculating process to identify the polymer swelling with the calculated S-PSP, or when the S-Distance calculated in step i) is identical to or smaller than the cut-off value, repeating steps b) and c) with a modification of the HSP adjusted in step b) until the S-Distance is larger than the cut-off value to identify the polymer swelling with the calculated S-PSP:

S-Distance=|Max-S-PSP−Min-S-PSP| Equation 4

wherein S-PSP stands for Solvent-Polymer Swelling Parameter, and Max-S-PSP and Min-S-PSP represents maximum and minimum values among the S-PSP values for N different solvents, respectively.

In detail, the cut-off value of step c) is imparted with no particular limitations so long as it is a real number larger than zero. That is, only when being larger than the predetermined cut-off value, the Solvent-Polymer Swelling Parameter Distance (S-Distance) is regarded as definitely elucidating polymer swelling. More concretely, the cut-off value is defined as a range in which a polymer undergoes swelling, and a cult-off value closer to zero represents a narrower range in which polymer swelling can occur. In a preferable embodiment of the present invention, the cut-off value is a real number corresponding to 20% to 40% of the maximum value among the Solvent-Polymer Swelling Parameters (S-PSPs) for N different solvents.

Also, contemplated according to the present invention is a system for calculating an evaluation index of polymer swelling, using the method of calculating an evaluation index of polymer swelling.

In detail, the system comprises:

an evaluation module for receiving data obtained by performing a swelling experiment with N different solvents to assess a degree of swelling of a polymer to be dissolved;

a data input module for receiving data obtained by calculating a Solvent-Polymer Swelling Parameter (S-PSP), based on Hansen Solubility Parameters (HSPs) adjusted for the N different solvents employed in the swelling experiment of the evaluation module; and

an identification module for receiving data obtained by calculating a Solvent-Polymer Swelling Parameter Distance (S-Distance) with regard to the N different solvents that are given the calculated S-PSP of the data input module to identify polymer swelling.

The evaluation module that assesses the degree of swelling of a polymer comprises measuring an increment of volume or weight of the polymer that swells due to the penetration of the solvent into the polymer.

In greater detail, the degree of swelling of a polymer may be measured as a relative amount of polymer swelling. The relative amount of polymer swelling may be a weight increment of the polymer upon swelling in each of the N different solvents, divided by the highest increment thereamong.

Furthermore, the number N of solvents used in the evaluation module is not specifically limited so long as it is a natural number larger than zero. In a preferable embodiment, the number N is an integer of 3 to 20.

In the present invention, the calculating of a Solvent-Polymer Swelling Parameter (S-PSP) of the data input module is performed using the following Equations 1 to 3:

S-PSP{Ai}=(a0x(ADJ_D{Ai})^{b0}+a1x(ADJ_P{Ai})^{b1}+a2x(ADJ_H{Ai})^{b2})^{c} Equation 1

ADJ_D{Ai}=F(D{Ai}),ADJ_P{Ai}=F(P{Ai}),ADJ_H{Ai}=F(H{Ai}) Equation 2

F(X)=d0×exp(X/d1) or F(X)=d2×log₁₀((X+1)/d3) Equation 3

In Equation 1, Ai represents an i^{th} solvent of the N different solvents used in the swelling experiment; a0, a1, and a2 are each real numbers; b0, b1, and b2 are each real numbers; and c is a real number. In Equation 2, ADJ_D{Ai}, ADJ_P{Ai}, and ADJ_H{Ai} each represent adjusted Hansen Solubility Parameters wherein D{Ai}, P{Ai}, and H{Ai} are respectively solubility parameters generated by non-polar dispersion, by polar energy due to a permanent dipole moment, and by energy within hydrogen bonds, respectively, for a certain solvent Ai. In Equation 3, F(X) is a function for adjusting an HSP for solvent Ai, and d0, d1, d2, and d3 are each real numbers. In the equations, preferably, b0 is a real number of 0 to 3.5, b1 is a real number of 0 to 5.0, and b2 is a real number of 0 to 4.0.

In the identification module, the calculating of Solvent-Polymer Swelling Parameter Distance (S-Distance) to identify polymer swelling comprises:

i) calculating a Solvent-Polymer Swelling Distance (S-Distance) according to the following Equation 4; and

ii) when the S-Distance calculated in step i) is larger than a cut-off value (a real number larger than zero), stopping the calculating process to identify the polymer swelling with the calculated S-PSP, or when the S-Distance calculated in step i) is identical to or smaller than the cut-off value, repeating steps b) and c) with a modification of the HSP adjusted in step b) until the S-Distance is larger than the cut-off value to identify the polymer swelling with the calculated S-PSP:

S-Distance=|Max-S-PSP−Min-S-PSP| Equation 4

wherein S-PSP stands for Solvent-Polymer Swelling Parameter, and Max-S-PSP and Min-S-PSP represents maximum and minimum values among the S-PSP values for N different solvents, respectively.

The cut-off value is defined as a range in which a polymer undergoes swelling, and a cult-off value closer to zero represents a narrower range in which polymer swelling can occur. In a preferable embodiment of the present invention, the cut-off value is a real number corresponding to 20% to 40% of the maximum value among the Solvent-Polymer Swelling Parameters (S-PSPs) for N different solvents.

As used herein, the term “module” refers to a unit for processing at least one function or operation and can be realized by hardware, software, or a combination thereof.

### MODE FOR INVENTION

Below, the present invention will be explained in greater detail with reference to the following embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method within the scope of these claims and their equivalents be covered thereby.

### Example

The polymer swelling system used in the Example was as follows.

1. Polymer: polydimethyl siloxane (PDMS)

2. Solvents used in swelling experiments: n-hexane, methylethyl ketone (MEK), and propylene glycol monomethyl ether acetate (PGMEA)

### 1. Swelling Experiment on Subject Polymer

The subject polymer, polydimethyl siloxane (PDMS) was subjected to a swelling experiment with the three solvents. In this regard, the polymer sample was soaked in each of the solvents for 20 to 30 min, followed by measuring weight increments of the subject polymer. Of the measurements, the greatest was detected upon swelling in n-hexane while the smallest was upon swelling in propylene glycol monomethyl ether acetate (PGMEA). The results are summarized in Table 1, below.

TABLE 1

| | Relative Swelling Amount of |
|---|---|
| | Subject Polymer PDMS |
| n-Hexane | 1.000 |
| MEK | 0.524 |
| PGMEA | 0.255 |

In Table 1, the relative swelling amounts of polydimethyl siloxane (PDMS) was obtained by dividing the weight increments of the polymer in individual solvents with the weight increment in the solvent n-hexane. Hence, the relative swelling amounts were measured to be 1.000 for n-hexane, and 0.255 for propylene glycol monomethyl ether acetate (PGMEA).

### 2. Calculation of S-PSP for N Different Solvents Used in Experiment (N=3)

With regard to the three solvents used in the polymer swelling experiment of Example 1-1, Solvent-Polymer Swelling Parameter was calculated according to Equations 1 and 2.

S-PSP{Ai}=(a0x(ADJ_D{Ai})^{b0}+a1x(ADJ_P{Ai})^{b1}+a2x(ADJ_H{Ai})^{b2})^{c} Equation 1

ADJ_D{Ai}=(0.5)×exp(D{Ai})/(6.0))

ADJ_P{Ai}=(0.8)×exp(P{Ai})/(2.1))

ADJ_H{Ai}=(1.2)×exp(H{Ai})/(2.2)) Equation 2

a0=2.0, a1=3.0, a2=3.0, b0=b1=b2=2.0, c=0.5

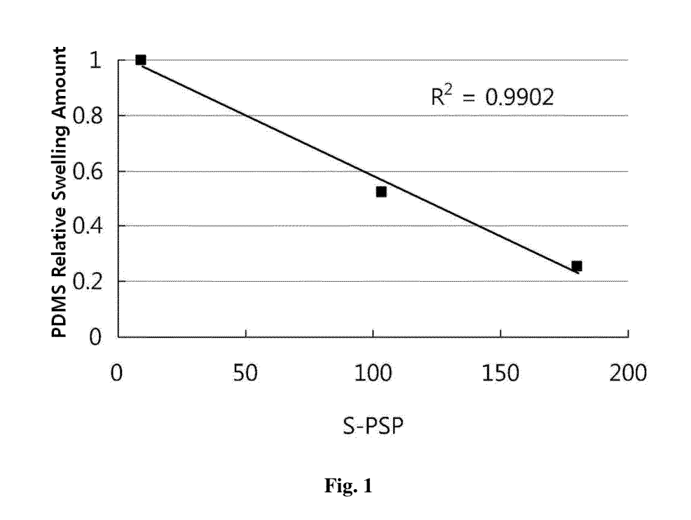

In Table 2, Solvent-Polymer Swelling Parameter (S-PSP) values calculated according to Equations 1 and 2 are given. As is understood from the data, the relative swelling amount tended to decrease with an increase in S-PSP, which demonstrates high correlation between the S-PSP and the relative swelling amount of polymer. FIG. 1 is a graph illustrating correlation between the Solvent-Polymer Swelling Parameter (S-PSP) and the relative swelling amount of polymer in accordance with an embodiment of the present invention, with a coefficient of determinant, R2 (R-square), set to be 0.9902.

TABLE 2

| | | Relative Swelling Amount of |
|---|---|---|
| | S-PSP | Subject Polymer PDMS |
| n-Hexane | 8.833 | 1.000 |
| MEK | 103.365 | 0.524 |
| PGMEA | 180.144 | 0.255 |

### 3. Calculation of Solvent-Polymer Swelling Parameter Distance

Solvent-Polymer Swelling Parameter Distance (S-Distance) for the three solvents was calculated according to Equation 4.

S-Distance=|Max-S-PSP−Min-S-PSP| Equation 4

Applying the measurements to Equation 4, S-Distance=|180.144−8.833|=171.311.

If a cut-off of 50.0 was set, the S-Distance was larger than the cut-off, thus meeting a requirement for polymer swelling. Consequently, the S-PSP calculated according to the method of the present invention can be useful for quantitatively evaluating polymer swelling, as proven in the experiment.

1. A method of calculating an evaluation index of polymer swelling, the method comprising:
a) performing a swelling experiment with N different solvents to assess a degree of swelling of a polymer to be dissolved;
b) calculating a Solvent-Polymer Swelling Parameter (S-PSP), based on Hansen Solubility Parameters (HSPs) adjusted for the N different solvents employed in the swelling experiment of step a); and
c) calculating a Solvent-Polymer Swelling Parameter Distance (S-Distance) with regard to the N different solvents that are given the calculated S-PSP of step b) to identify polymer swelling.

2. The method of claim 1, wherein the assessing of a degree of swelling of a polymer of step a) comprises measuring an increment of volume or weight of the polymer that swells due to the penetration of the solvent into the polymer.

3. The method of claim 2, wherein the degree of swelling of a polymer is measured as a relative amount of polymer swelling, the relative amount of polymer swelling being defined as a weight increment of the polymer on swelling in each of the N different solvents, divided by the highest increment thereamong.

4. The method of claim 1, wherein the N of step 3 is an integer of 3 to 20.

5. The method of claim 1, wherein the calculating of a Solvent-Polymer Swelling Parameter (S-PSP) of step b) is performed using the following Equations 1 to 3:
S-PSP{Ai}=(a0x(ADJ_D{Ai})^{b0}+a1x(ADJ_P{Ai})^{b1}+a2x(ADJ_H{Ai})^{b2})^{c} Equation 1
wherein,
Ai represents an i^{th} solvent of the N different solvents used in the swelling experiment;
a0, a1, and a2 are each real numbers;
b0, b1, and b2 are each real numbers; and
c is a real number,
ADJ_D{Ai}=F(D{Ai}),ADJ_P{Ai}=F(P{Ai}),ADJ_H{Ai}=F(H{Ai}) Equation 2
wherein ADJ_D{Ai}, ADJ_P{Ai}, and ADJ_H{Ai} each represent adjusted Hansen Solubility Parameters wherein D{Ai}, P{Ai}, and H{Ai} are respectively solubility parameters generated by non-polar dispersion, by polar energy due to a permanent dipole moment, and by energy within hydrogen bonds, respectively, for a certain solvent Ai; and
F(X)=d0×exp(X/d1) or F(X)=d2×log₁₀((X+1)/d3) Equation 3
wherein,
F(X) is a function for adjusting an HSP for solvent Ai, and d0, d1, d2, and d3 are each real numbers.

6. The method of claim 5, wherein b0 is a real number of 0 to 3.5, b1 is a real number of 0 to 5.0, and b2 is a real number of 0 to 4.0.

7. The method of claim 1, wherein
the calculating of Solvent-Polymer Swelling Parameter Distance (S-Distance) to identify polymer swelling comprises:
i) calculating a Solvent-Polymer Swelling Distance (S-Distance) according to the following Equation 4; and
ii) when the S-Distance calculated in step i) is larger than a cut-off value (a real number larger than zero), stopping the calculating process to identify the polymer swelling with the calculated S-PSP, or when the S-Distance calculated in step i) is identical to or smaller than the cut-off value, repeating steps b) and c) with a modification of the HSP adjusted in step b) until the S-Distance is larger than the cut-off value to identify the polymer swelling with the calculated S-PSP:
S-Distance=|Max-S-PSP−Min-S-PSP| Equation 4
wherein S-PSP stands for Solvent-Polymer Swelling Parameter, and Max-S-PSP and Min-S-PSP represents maximum and minimum values among the S-PSP values for N different solvents, respectively.

8. The method of claim 7, wherein the cut-off value is a real number corresponding to 20% to 40% of the maximum value among the Solvent-Polymer Swelling Parameters (S-PSPs) for N different solvents.

9. A system for calculating an evaluation index of polymer swelling, comprising:
an evaluation module for receiving data obtained by performing a swelling experiment with N different solvents to assess a degree of swelling of a polymer to be dissolved;
a data input module for receiving data obtained by calculating a Solvent-Polymer Swelling Parameter (S-PSP), based on Hansen Solubility Parameters (HSPs) adjusted for the N different solvents employed in the swelling experiment of the evaluation module; and
an identification module for receiving data obtained by calculating Solvent-Polymer Swelling Parameter Distance (S-Distance) with regard to the N different solvents that are given the calculated S-PSP of the data input module to identify polymer swelling.

10. The system of claim 9, wherein the assessing of a degree of swelling of a polymer of the evaluation module is achieved by measuring an increment of volume or weight of the polymer that swells due to the penetration of the solvent into the polymer.

11. The system of claim 10, wherein the degree of swelling of a polymer is measured as a relative amount of polymer swelling, the relative amount of polymer swelling being defined as a weight increment of the polymer on swelling in each of the N different solvents, divided by the highest increment thereamong.

12. The system of claim 9, wherein the N of step 3 is an integer of 3 to 20.

13. The method of claim 9, wherein the calculating of a Solvent-Polymer Swelling Parameter (S-PSP) of the data input module is performed using the following Equations 1 to 3:
S-PSP{Ai}=(a0x(ADJ_D{Ai})^{b0}+a1x(ADJ_P{Ai})^{b1}+a2x(ADJ_H{Ai})^{b2})^{c} Equation 1
ADJ_D{Ai}=F(D{Ai}),ADJ_P{Ai}=F(P{Ai}),ADJ_H{Ai}=F(H{Ai}) Equation 2
F(X)=d0×exp(X/d1) or F(X)=d2×log₁₀((X+1)/d3) Equation 3
wherein,
Ai represents an i^{th} solvent of the N different solvents used in the swelling experiment,
a0, a1, and a2 are each real numbers,
b0, b1, and b2 are each real numbers, and c is a real number,
ADJ_D{Ai}, ADJ_P{Ai}, and ADJ_H{Ai} each represent adjusted Hansen Solubility Parameters wherein D{Ai}, P{Ai}, and H{Ai} are respectively solubility parameters generated by non-polar dispersion, by polar energy due to a permanent dipole monument, and by energy within hydrogen bonds, respectively, for a certain solvent Ai, and
F(X) is a function for adjusting an HSP for solvent Ai, and d0, d1, d2, and d3 are each real numbers.

14. The system of claim 13, wherein b0 is a real number of 0 to 3.5, b1 is a real number of 0 to 5.0, and b2 is a real number of 0 to 4.0.

15. The system of claim 9, wherein
the calculating of Solvent-Polymer Swelling Parameter Distance (S-Distance) to identify polymer swelling in the identification module comprises:
i) calculating a Solvent-Polymer Swelling Distance (S-Distance) according to the following Equation 4; and
ii) when the S-Distance calculated in step i) is larger than a cut-off value (a real number larger than zero), stopping the calculating process to identify the polymer swelling with the calculated S-PSP, or when the S-Distance calculated in step i) is identical to or smaller than the cut-off value, repeating steps b) and c) with a modification of the HSP adjusted in step b) until the S-Distance is larger than the cut-off value to identify the polymer swelling with the calculated S-PSP:
S-Distance=|Max-S-PSP−Min-S-PSP| Equation 4
wherein S-PSP stands for Solvent-Polymer Swelling Parameter, and Max-S-PSP and Min-S-PSP represents maximum and minimum values among the S-PSP values for N different solvents, respectively.

16. The system of claim 15, wherein the cut-off value is a real number corresponding to 20% to 40% of the maximum value among the Solvent-Polymer Swelling Parameters (S-PSPs) for N different solvents.
